SEQUENCE LISTING (37 sequences)

SEQ ID NO: 1 - DNA, 23 bp, Artificial Sequence
  source: /note=“Description of Artificial Sequence Syntheticprimer”
gaatataaacttgtggtagttgg

SEQ ID NO: 2 - DNA, 23 bp, Artificial Sequence
  source: /note=“Description of Artificial Sequence Syntheticprimer”
aatgactgaatataaacttgtgg

SEQ ID NO: 3 - DNA, 23 bp, Artificial Sequence
  source: /note=“Description of Artificial Sequence Syntheticprimer”
aatataaacttgtggtagttgga

SEQ ID NO: 4 - DNA, 19 bp, Artificial Sequence
  source: /note=“Description of Artificial Sequence Syntheticprimer”
gtatcgtcaaggcactctt

SEQ ID NO: 5 - DNA, 18 bp, Artificial Sequence
  source: /note=“Description of Artificial Sequence Syntheticprimer”
tagctgtatcgtcaaggc

SEQ ID NO: 6 - DNA, 58 bp, Homo sapiens
gaatataaacttgtggtagttggagctggtggcgtaggcaagagtgccttgacgatac

SEQ ID NO: 7 - DNA, 65 bp, Homo sapiens
aatgactgaatataaacttgtggtagttggagctggtggcgtaggcaagagtgccttgacgatac

SEQ ID NO: 8 - DNA, 70 bp, Homo sapiens
aatgactgaatataaacttgtggtagttggagctggtggcgtaggcaagagtgccttgacgatacagcta

SEQ ID NO: 9 - DNA, 57 bp, Homo sapiens
aatataaacttgtggtagttggagctggtggcgtaggcaagagtgccttgacgatac

SEQ ID NO: 10 - DNA, 58 bp, Artificial Sequence
  source: /note=“Description of Artificial Sequence Syntheticoligonucleotide”
gaatataaacttgtggtagttggagcttgtggcgtaggcaagagtgccttgacgatac

SEQ ID NO: 11 - DNA, 58 bp, Artificial Sequence
  source: /note=“Description of Artificial Sequence Syntheticoligonucleotide”
gaatataaacttgtggtagttggagctgttggcgtaggcaagagtgccttgacgatac

SEQ ID NO: 12 - DNA, 58 bp, Artificial Sequence
  source: /note=“Description of Artificial Sequence Syntheticoligonucleotide”
gaatataaacttgtggtagttggagctagtggcgtaggcaagagtgccttgacgatac

SEQ ID NO: 13 - DNA, 58 bp, Artificial Sequence
  source: /note=“Description of Artificial Sequence Syntheticoligonucleotide”
gaatataaacttgtggtagttggagctgatggcgtaggcaagagtgccttgacgatac

SEQ ID NO: 14 - DNA, 58 bp, Artificial Sequence
  source: /note=“Description of Artificial Sequence Syntheticoligonucleotide”
gaatataaacttgtggtagttggagctggtgacgtaggcaagagtgccttgacgatac

SEQ ID NO: 15 - DNA, 58 bp, Artificial Sequence
  source: /note=“Description of Artificial Sequence Syntheticoligonucleotide”
gaatataaacttgtggtagttggagctcgtggcgtaggcaagagtgccttgacgatac

SEQ ID NO: 16 - DNA, 58 bp, Artificial Sequence
  source: /note=“Description of Artificial Sequence Syntheticoligonucleotide”
gaatataaacttgtggtagttggagctggtcgcgtaggcaagagtgccttgacgatac

SEQ ID NO: 17 - DNA, 65 bp, Artificial Sequence
  source: /note=“Description of Artificial Sequence Syntheticoligonucleotide”
aatgactgaatataaacttgtggtagttggagcttgtggcgtaggcaagagtgccttgacgatac

SEQ ID NO: 18 - DNA, 65 bp, Artificial Sequence
  source: /note=“Description of Artificial Sequence Syntheticoligonucleotide”
aatgactgaatataaacttgtggtagttggagctgttggcgtaggcaagagtgccttgacgatac

SEQ ID NO: 19 - DNA, 65 bp, Artificial Sequence
  source: /note=“Description of Artificial Sequence Syntheticoligonucleotide”
aatgactgaatataaacttgtggtagttggagctagtggcgtaggcaagagtgccttgacgatac

SEQ ID NO: 20 - DNA, 65 bp, Artificial Sequence
  source: /note=“Description of Artificial Sequence Syntheticoligonucleotide”
aatgactgaatataaacttgtggtagttggagctgatggcgtaggcaagagtgccttgacgatac

SEQ ID NO: 21 - DNA, 65 bp, Artificial Sequence
  source: /note=“Description of Artificial Sequence Syntheticoligonucleotide”
aatgactgaatataaacttgtggtagttggagctggtgacgtaggcaagagtgccttgacgatac

SEQ ID NO: 22 - DNA, 65 bp, Artificial Sequence
  source: /note=“Description of Artificial Sequence Syntheticoligonucleotide”
aatgactgaatataaacttgtggtagttggagctcgtggcgtaggcaagagtgccttgacgatac

SEQ ID NO: 23 - DNA, 65 bp, Artificial Sequence
  source: /note=“Description of Artificial Sequence Syntheticoligonucleotide”
aatgactgaatataaacttgtggtagttggagctggtcgcgtaggcaagagtgccttgacgatac

SEQ ID NO: 24 - DNA, 70 bp, Artificial Sequence
  source: /note=“Description of Artificial Sequence Syntheticoligonucleotide”
aatgactgaatataaacttgtggtagttggagcttgtggcgtaggcaagagtgccttgacgatacagcta

SEQ ID NO: 25 - DNA, 70 bp, Artificial Sequence
  source: /note=“Description of Artificial Sequence Syntheticoligonucleotide”
aatgactgaatataaacttgtggtagttggagctgttggcgtaggcaagagtgccttgacgatacagcta

SEQ ID NO: 26 - DNA, 70 bp, Artificial Sequence
  source: /note=“Description of Artificial Sequence Syntheticoligonucleotide”
aatgactgaatataaacttgtggtagttggagctagtggcgtaggcaagagtgccttgacgatacagcta

SEQ ID NO: 27 - DNA, 70 bp, Artificial Sequence
  source: /note=“Description of Artificial Sequence Syntheticoligonucleotide”
aatgactgaatataaacttgtggtagttggagctgatggcgtaggcaagagtgccttgacgatacagcta

SEQ ID NO: 28 - DNA, 70 bp, Artificial Sequence
  source: /note=“Description of Artificial Sequence Syntheticoligonucleotide”
aatgactgaatataaacttgtggtagttggagctggtgacgtaggcaagagtgccttgacgatacagcta

SEQ ID NO: 29 - DNA, 70 bp, Artificial Sequence
  source: /note=“Description of Artificial Sequence Syntheticoligonucleotide”
aatgactgaatataaacttgtggtagttggagctcgtggcgtaggcaagagtgccttgacgatacagcta

SEQ ID NO: 30 - DNA, 70 bp, Artificial Sequence
  source: /note=“Description of Artificial Sequence Syntheticoligonucleotide”
aatgactgaatataaacttgtggtagttggagctggtcgcgtaggcaagagtgccttgacgatacagcta

SEQ ID NO: 31 - DNA, 57 bp, Artificial Sequence
  source: /note=“Description of Artificial Sequence Syntheticoligonucleotide”
aatataaacttgtggtagttggagcttgtggcgtaggcaagagtgccttgacgatac

SEQ ID NO: 32 - DNA, 57 bp, Artificial Sequence
  source: /note=“Description of Artificial Sequence Syntheticoligonucleotide”
aatataaacttgtggtagttggagctgttggcgtaggcaagagtgccttgacgatac

SEQ ID NO: 33 - DNA, 57 bp, Artificial Sequence
  source: /note=“Description of Artificial Sequence Syntheticoligonucleotide”
aatataaacttgtggtagttggagctagtggcgtaggcaagagtgccttgacgatac

SEQ ID NO: 34 - DNA, 57 bp, Artificial Sequence
  source: /note=“Description of Artificial Sequence Syntheticoligonucleotide”
aatataaacttgtggtagttggagctgatggcgtaggcaagagtgccttgacgatac

SEQ ID NO: 35 - DNA, 57 bp, Artificial Sequence
  source: /note=“Description of Artificial Sequence Syntheticoligonucleotide”
aatataaacttgtggtagttggagctggtgacgtaggcaagagtgccttgacgatac

SEQ ID NO: 36 - DNA, 57 bp, Artificial Sequence
  source: /note=“Description of Artificial Sequence Syntheticoligonucleotide”
aatataaacttgtggtagttggagctcgtggcgtaggcaagagtgccttgacgatac

SEQ ID NO: 37 - DNA, 57 bp, Artificial Sequence
  source: /note=“Description of Artificial Sequence Syntheticoligonucleotide”
aatataaacttgtggtagttggagctggtcgcgtaggcaagagtgccttgacgatac

(19) United States
(12) Patent Application Publication
(10) Pub. No.: US 2011/0207143 A1
(43) Pub. Date: Aug. 25, 2011

(54) DIAGNOSTIC TEST FOR MUTATIONS IN CODONS 12-13 OF HUMAN K-RAS

(75) Inventors: Mark A. Hayden, Ingleside, IL (US); Thomas G. Laffler, Libertyville, IL (US)

(73) Assignee: Abbott Laboratories, Abbott Park, IL (US)

(21) Appl. No.: 13/126,362

(22) PCT Filed: Dec. 18, 2009
(86) PCT No.: PCT/US2009/068778

§ 371 Date: Apr. 27, 2011

Related U.S. Application Data
(60) Provisional application No. 61/139,413, filed on Dec. 19, 2008.

(51) Int. Cl. C12Q 1/68

(52) U.S. Cl. 435/6.12

(57) ABSTRACT

The invention is directed to compositions, methods and kits for diagnosing cancers and tumors correlated with mutations in codons 12 and 13 of human K-RAS using primers that amplify target sequences. The amplified target sequences are then analyzed by any number of mass spectrometric techniques, which data are queried against a database of base composition signatures of K-RAS mutations in codons 12 and 13.

### FIELD OF THE INVENTION

The present invention is generally directed to the detection of mutations in codons 12-13 of human K-RAS, which are markers for various cancers, using molecular tools.

### GOVERNMENT SUPPORT

Not applicable.

### COMPACT DISC FOR SEQUENCE LISTINGS AND TABLES

Not applicable.

### BACKGROUND OF THE INVENTION

K-RAS and Cancer

Mutations in the K-RAS (derived from “RAt Sarcoma” virus; also known as K-RAS2) gene correlates with a wide variety of cancers. The K-RAS gene encodes the human cellular homolog of a transforming gene isolated from the Kirsten rat sarcoma virus. Mutation of the K-RAS gene at codons 12 and 13 leads to functional modification of the encoded p21-protein. K-RAS mutations are known to occur in roughly 90% of pancreatic cancers, 50% of colorectal cancers and 30% of non-small cell lung cancers, and its mutation profile has revealed that about 85% of mutations occur at codons 12 and 13 (Samowitz et al., 2000). Identifying K-RAS gene mutations is used as a useful tool in cancer diagnosis, e.g., pancreatic, colorectal and non-small cell lung cancers, and studies have shown correlations with various tumor phenotypes (Andreyev et al., 2001; Brink et al., 2003; Samowitz et al., 2000).

The RAS oncogene family is well-characterized (Barbacid, 1987); (Bos, 1989). The RAS gene family encodes immunologically related proteins having molecular weights of 21,000 (p21) (Ellis et al., 1981); (Papageorge et al., 1982). These are guanosine diphosphate/guanosine triphosphate (GDP/GTP)-binding proteins that act as intracellular signal transducers. This family has at least 3 members (1) H-RAS in the Harvey strain of murine sarcoma virus (Ellis et al., 1981), (2) K-RAS and Kirsten murine sarcoma virus (Ellis et al., 1981), and (3) N-RAS identified by low stringency hybridization to H-RAS (Shimizu et al., 1983). The wild-type proteins play vital roles in normal tissue signaling, including proliferation, differentiation, and senescence, while mutated RAS genes play roles in many human cancers (Kranenburg, 2005).

K-RAS appears to be one of the most commonly activated oncogenes, with 17-25% of all human tumors harboring an activating K-RAS mutation (Kranenburg, 2005). Critical regions in context of oncogenic activation besides codons 12 and 13 include codons 59, 61 and 63 (Grimmond et al., 1992). The mutations result in RAS accumulating in the active GTP-bound state, apparently resulting from impaired intrinsic GTPase activity, thus conferring resistance to GTPase activating proteins (Zenker et al., 2007).

Examples of studies that uncovered K-RAS mutations in codons 12 and 13 are shown in Table 1.

TABLE 1
Non-limiting examples of codon 12 and 13 mutations in K-RAS

| Codon | Reference | Notes |
|---|---|---|
| 12 | (Rodenhuis et al., 1987) | Found in 5 of 10 adenocarcinomas |
| 12 | (Yanez et al., 1987) | Found in 4 of 16 colon cancers, 2 of 27 lung cancers and 1 of 8 breast cancers |
| 12 | (Almoguera et al., 1988; Hruban et al., | 90% of patients suffering from pancreatic carcinomas harbor a point mutation |
| | 1993; Smit et at., 1988) (Laghi et al., 2002) | in KRAS; most of these mutations reside in codon 12. |
| 12 | (Burmer and Loeb, 1989) | 26 of 40 colon carcinomas, and 9 of 12 adenomas carried mutations in codon |
| | | 12. |
| 12 | (Lee et al., 1995) | Found mutations in some gastric cancers, with mutation rates increased in |
| | | tumors in the upper third of the stomach compared to the other parts of the |
| | | stomach. |
| 12, 13 | (Otori et al., 1997) | 9 of 19 hyperplastic colorectal polyps, and 5 of 9 colorectal adenomas showed |
| | | mutations in codons 12 and 13. |
| 12, 13 | (Nikiforova et al., 2003) | Found high rates of mutations in thyroid follicular carcinomas and Hurthle cell |
| | | thyroid tumors. |
| 13 | (Liu et al., 1987) | Found mutations in codon 13 in patients with preleukemia and myelodysplastic |
| | | syndrome. |
| 12, 13 | (Bezieau et al., 2001) | Uncovered codon 12 and 13 mutations in patients with multiple myeloma and |
| | | related disorders. |

Prior Methods of Determining K-RAS Codon 12-13 Mutations

Typical molecular assays for mutations at codons 12-13 of human K-RAS gene depend on a step to detect a polymorphism from wild-type. For example, a test sample containing nucleic acids suspected of harboring a mutation in codon 12 or 13 of K-RAS is analyzed by a variety of techniques including RNA/RNA or RNA/DNA duplex base mis-match detection followed by gel electrophoresis; electrophoretic mobility assays, such as single-strand conformation polymorphism (SSCP) assays that detect differences in electrophoretic mobility between mutant and wild-type polynucleotides on a gel, or similarly, denaturing gradient gel electrophoresis (DGGE), which involves the extra step of adding a GC clamp to the target sequence. Other examples include selective oligonucleotide hybridization, selective nucleic acid amplification techniques, and selective primer extension assays. Selective PCR amplification approaches are also used, wherein primers carry the mutation of interest in the center of the molecule (thus requiring a different primer for each mutation), or at the 3′ end of the primer (ARMS), etc. Alternatively, the sequence from the test sample is compared to the wild-type K-RAS sequence (thus requiring sequencing), or assaying for a restriction fragment length polymorphism (RFLP) (thus requiring identifying useful RFLPs, and involving extra steps, such as restriction nuclease digestion and fragment length detection).

### Nucleic Acid-Based Molecular Diagnostics

Molecular diagnostics have been championed for identifying nucleic acids. Polymerase chain reaction (PCR)-based diagnostics, wherein target polynucleotide sequences are amplified in vitro and then detected, have been successfully developed for a wide variety of nucleic acids, specific to disease, conditions, and pathogens.

The principal shortcomings of applying PCR assays to the clinical setting include the inability to eliminate background DNA contamination and interference with the PCR amplification by competing substrates. Despite significant progress, contamination of test samples remains problematic, and methods directed towards eliminating exogenous sources of DNA often also result in significant diminution in assay sensitivity. Although simple DNA sequencing can be performed to identify and characterize PCR products, sequencing and the subsequent analysis is laborious and time-consuming.

Mass spectrometric techniques, such as high resolution electrospray ionization-Fourier transform-ion cyclotron resonance mass spectrometry (ESI-FT-ICR MS), can be used for quick PCR product detection and characterization. Accurate measurement of the exact mass combined with knowledge of the number of at least one nucleotide allows for calculating the total base composition for PCR duplex products of approximately 100 base pairs (Muddiman and Smith, 1998). For example, Aaserud et al demonstrated that accurate mass measurements obtained by high-performance mass spectrometry can be used to derive base compositions from double-stranded synthetic DNA constructs using the mathematical constraints imposed by the complementary nature of the two strands (Aaserud et al., 1996). Muddiman et al. developed an algorithm that allowed for deriving unambiguous base compositions from the exact mass measurements of the complementary single-stranded oligonucleotides (Muddiman et al., 1997). Wunschel et al showed that PCR products amplified from templates differing by a single nucleotide can be resolved and identified using ESI-FTICR at the 89-bp level in PCR product amplified from a 16/23S rDNA interspace region from Bacillus cereus (Wunschel et al., 1998). Electrospray ionization-Fourier transform-ion cyclotron resistance (ESI-FT-ICR) MS can be used to determine the mass of double-stranded, 500 base-pair PCR products via the average molecular mass (Hurst et al., 1996). The use of matrix-assisted laser desorption ionization-time of flight (MALDI-TOF) mass spectrometry for characterizing PCR products has also been exploited (Muddiman et al., 1999).

Examples of mass spectrometric analysis of polynucleotides include:

U.S. Pat. No. 5,965,363 discloses methods for screening nucleic acids for polymorphisms by analyzing amplified target nucleic acids using mass spectrometric techniques and procedures for improving mass resolution and mass accuracy of these methods.

WO 99/14375 describes methods, PCR primers and kits for use in analyzing preselected DNA tandem nucleotide repeat alleles by mass spectrometry.

WO 98/12355 discloses methods of determining the mass of a target nucleic acid by mass spectrometric analysis, by cleaving the target nucleic acid to reduce its length, making the target single-stranded and using MS to determine the mass of the single-stranded shortened target. Also disclosed are methods of preparing a double-stranded target nucleic acid for MS analysis comprising amplification of the target nucleic acid, binding one of the strands to a solid support, releasing the second strand and then releasing the first strand which is then analyzed by MS. Kits for target nucleic acid preparation are also provided.

PCT WO97/33000 discloses methods for detecting mutations in a target nucleic acid by non-randomly fragmenting the target into a set of single-stranded nonrandom length fragments and determining their masses by MS.

U.S. Pat. No. 5,605,798 describes a fast and highly accurate mass spectrometer-based process for detecting the presence of a particular nucleic acid in a biological sample for diagnostic purposes.

WO 98/21066 describes processes for determining the sequence of a particular target nucleic acid by mass spectrometry. Processes for detecting a target nucleic acid present in a biological sample by PCR amplification and mass spectrometry detection are disclosed, as are methods for detecting a target nucleic acid in a sample by amplifying the target with primers that contain restriction sites and tags, extending and cleaving the amplified nucleic acid, and detecting the presence of extended product, wherein the presence of a DNA fragment of a mass different from wild-type is indicative of a mutation. Methods of sequencing a nucleic acid via mass spectrometry methods are also described.

WO 97/37041, WO 99/31278 and U.S. Pat. No. 5,547,835 describe methods of sequencing nucleic acids using mass spectrometry. U.S. Pat. Nos. 5,622,824, 5,872,003 and 5,691,141 describe methods, systems and kits for exonuclease-mediated mass spectrometric sequencing.

U.S. Pat. Nos. 7,217,510, 7,108,974, 7,255,992, 7,226,739 and US 2004/0219517 describe methods and compositions for exploiting base composition signatures; in these cases, identifying pathogens. The base composition signature, the exact base composition determined from the molecular mass of the amplified product, is determined by first determining the molecular mass of the amplification product by mass spectrometry, after which the base composition is determined from the molecular mass. At least one pair of oligonucleotide primers, wherein the pair hybridizes to two distinct conserved regions of a nucleic acid encoding a ribosomal RNA, wherein the two distinct conserved regions flank a variable nucleic acid region that when amplified creates a base composition “signature” that is characteristic of a pathogen. The pathogen is determined by matching the base composition signature to those stored in a database.

### SUMMARY OF THE INVENTION

In a first aspect, the invention is directed to methods of identifying the presence or absence of mutations in codon 12 or 13 of human K-RAS in a test sample, comprising:

providing a test sample;

forming a reaction mixture comprising a primer pair set selected from the group consisting of set A, B, C, and D, wherein:
set A comprises a forward primer comprising a nucleic acid sequence of SEQ ID NO:1, and a reverse primer comprising a nucleic acid sequence of SEQ ID NO:4;
set B comprises a forward primer comprising a nucleic acid sequence of SEQ ID NO:2, and a reverse primer comprising a nucleic acid sequence of SEQ ID NO:4;
set C comprises a forward primer comprising a nucleic acid sequence of SEQ ID NO:2, and a reverse primer comprising a nucleic acid sequence of SEQ ID NO:5;
set D comprises a forward primer comprising a nucleic acid sequence of SEQ ID NO:3, and a reverse primer comprising a nucleic acid sequence of SEQ ID NO:4;

subjecting the mixture to amplification conditions to generate an amplification product;

determining the molecular mass of the amplification product; and

comparing the molecular mass of the amplification product to calculated or measured molecular masses of target sequences in a database to identify the presence or absence mutations in codon 12 or 13 of human K-RAS.

In a second aspect, the invention is directed to methods of identifying the presence or absence of mutations in codon 12 or 13 of human K-RAS in a test sample, comprising:

providing a test sample;

forming a reaction mixture comprising a primer pair set selected from the group consisting of set A, B, C, and D, wherein:
set A comprises a forward primer comprising a nucleic acid sequence of SEQ ID NO:1, and a reverse primer comprising a nucleic acid sequence of SEQ ID NO:4;
set B comprises a forward primer comprising a nucleic acid sequence of SEQ ID NO:2, and a reverse primer comprising a nucleic acid sequence of SEQ ID NO:4;
set C comprises a forward primer comprising a nucleic acid sequence of SEQ ID NO:2, and a reverse primer comprising a nucleic acid sequence of SEQ ID NO:5;
set D comprises a forward primer comprising a nucleic acid sequence of SEQ ID NO:3, and a reverse primer comprising a nucleic acid sequence of SEQ ID NO:4;

subjecting the mixture to amplification conditions to generate an amplification product;

determining the base composition of the amplification product; and comparing the base composition of the amplification product to calculated or measured base compositions of target sequences in a database to identify the presence or absence of mutations in codon 12 or 13 of human K-RAS.

In either of the methods of the first and second aspects, identifying the target sequence does not comprise sequencing of the amplification product, and the mass spectrometry can be Fourier transform ion cyclotron resonance mass spectrometry (FT-ICR-MS) or time of flight mass spectrometry (TOF-MS), such as electrospray ionization time of flight mass spectrometry (ESI-TOF). Additionally, in either of the first and second aspects, the primer set can comprise at least one nucleotide analog, wherein the nucleotide analog is, for example, inosine, uridine, 2,6-diaminopurine, propyne C, and propyne T. The amplification products of both aspects can further comprise incorporating a molecular mass-modifying tag, such as an isotope of carbon, for example, ¹³C. Detecting mutations in codons 12 or 13 correlates with a cancer selected from the group consisting of colorectal, non-small cell lung, ovarian, bile duct, pancreatic, esophageal, breast, thyroid, and endometrial, or any other cancer or tumor that correlates with a mutation in codon 12 or 13 of K-RAS.

In a third aspect, the invention is directed to methods of identifying the presence or absence of mutations in codon 12 or 13 of human K-RAS in a test sample, comprising:

providing a test sample;

forming a reaction mixture comprising:

a primer pair set selected from the group consisting of set A, B, C, and D, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:4;
set B comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:4;
set C comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:5; and
set D comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4;

subjecting the mixture to amplification conditions to generate an amplification product;

determining the molecular mass of the amplification product; and comparing the molecular mass of the amplification product to calculated or measured molecular masses of target sequences in a database to identify the presence or absence of mutations in codon 12 or 13 of human K-RAS.

In a fourth aspect, the invention is directed to methods of identifying the presence or absence of mutations in codon 12 or 13 of human K-RAS in a test sample, comprising:

providing a test sample;

forming a reaction mixture comprising:

a primer pair set selected from the group consisting of set A, B, C, and D, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:4
set B comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:4;
set C comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:5; and
set D comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4;

subjecting the mixture to amplification conditions to generate an amplification product;

determining the base composition of the amplification product; and

comparing the base composition of the amplification product to calculated or measured base compositions of target sequences in a database to identify the presence or absence of mutations in codons 12 or 13 of human K-RAS, wherein K-RAS mutations are correlated with at least one cancer or tumor.

In either of the methods of the third and fourth aspects, identifying the target sequence does not comprise sequencing of the amplification product, and the mass spectrometry can be Fourier transform ion cyclotron resonance mass spectrometry (FT-ICR-MS) or time of flight mass spectrometry (TOF-MS), such as electrospray ionization time of flight mass spectrometry. Additionally, in either of the third and fourth aspects, the primer set can comprise at least one nucleotide analog, wherein the nucleotide analog is, for example, inosine, uridine, 2,6-diaminopurine, propyne C, and propyne T, and the reaction mixture comprises at least two primer sets. The amplification products of both aspects can further comprise incorporating a molecular mass-modifying tag. Detecting mutations in codons 12 or 13 correlates with a cancer selected from the group consisting of colorectal, non-small cell lung, ovarian, bile duct, pancreatic, esophageal, breast, thyroid, and endometrial, or any other cancer or tumor that correlates with a mutation in codon 12 or 13 of K-RAS.

In a fifth aspect, the invention is directed to kits, comprising a primer pair set selected from the group consisting of set A, B, C, and D, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:4
set B comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:4;
set C comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:5; and
set D comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4; and

amplification reagents.

In a sixth aspect, the invention is directed to kits, comprising a primer pair set selected from the group consisting of set A, B, C, and D, wherein:
set A comprises a forward primer comprising a nucleic acid sequence of SEQ ID NO:1, and a reverse primer comprising a nucleic acid sequence of SEQ ID NO:4;
set B comprises a forward primer comprising a nucleic acid sequence of SEQ ID NO:2, and a reverse primer comprising a nucleic acid sequence of SEQ ID NO:4;
set C comprises a forward primer comprising a nucleic acid sequence of SEQ ID NO:2, and a reverse primer comprising a nucleic acid sequence of SEQ ID NO:5;
set D comprises a forward primer comprising a nucleic acid sequence of SEQ ID NO:3, and a reverse primer comprising a nucleic acid sequence of SEQ ID NO:4; and

amplification reagents.

In a seventh aspect, the invention is directed to primer pair sets, wherein the primer pair set is one selected from the group consisting of set A, B, C, and D, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:4
set B comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:4;
set C comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:5; and
set D comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4.

In an eighth aspect, the invention is directed to primer pair sets, wherein the primer pair set is one selected from the group consisting of set A, B, C, and D, wherein:
set A comprises a forward primer comprising a nucleic acid sequence of SEQ ID NO:1, and a reverse primer comprising a nucleic acid sequence of SEQ ID NO:4;
set B comprises a forward primer comprising a nucleic acid sequence of SEQ ID NO:2, and a reverse primer comprising a nucleic acid sequence of SEQ ID NO:4;
set C comprises a forward primer comprising a nucleic acid sequence of SEQ ID NO:2, and a reverse primer comprising a nucleic acid sequence of SEQ ID NO:5; and
set D comprises a forward primer comprising a nucleic acid sequence of SEQ ID NO:3, and a reverse primer comprising a nucleic acid sequence of SEQ ID NO:4,

### BRIEF DESCRIPTION OF THE DRAWING

Not applicable.

### DETAILED DESCRIPTION

The present invention greatly simplifies the detection of mutations in K-RAS codons 12 and 13. After amplification of the target sequence using novel primer pair sets, the resulting fragment is analyzed by mass spectrometry for base composition, wherein the presence of mutations are indicated by specific alterations in base composition.

The invention accomplishes its significant advantages in part by exploiting spectrometric technologies. For example, ElectroSpray Injection Time-of-Flight Mass Spectrometry (ESI-MS) can be used to determine the exact base composition of amplicons generated by target amplification technologies such as the polymerase chain reaction (PCR). The disclosed invention exploits primer pairs (sets A, B, C, and D) that flank codons 12-13 of K-RAS. The primers are shown in Table 2, and the sequences they amplify are shown in Table 3. Table 4 shows non-limiting examples of mutations in codons 12 and 13, showing the nucleic acid change, as well as the resulting amino acid change. Tables 5-8 show the sequences that each primer set amplifies when the amplified nucleic acid carries the different mutations shown in Table 4.

The invention provides for novel sets of primer pairs. These primer pair sets can be used in various K-RAS mutation detection methods of the invention. In one embodiment, the invention provides and uses the novel primer pair of SEQ ID NOs:1 and 4 (Set A). In another embodiment, the invention provides and uses the novel primer pair of SEQ ID NOs:2 and 4 (Set B). In another embodiment, the invention provides and uses the primer pair of SEQ ID NOs:2 and 5 (Set C). In yet another embodiment, the invention provides and uses the primer pair of SEQ ID NOs:3 and 4 (Set D).

TABLE 2
Primer pair sets and sequences

| | | | SEQ | | |
|---|---|---|---|---|---|
| | | | ID | Length, | Tm, |
| Set | Primer | Sequence | NO | nt | ° C. |
| A | F-102 | gaatataaac ttgtggtagt | 1 | 23 | 61 |
| | (Forward) | tgg | | | |
| | R-141 | gtatcgtcaa ggcactctt | 4 | 19 | 61 |
| | (Reverse) | | | | |
| B | F-95 | aatgactgaa tataaacttg | 2 | 23 | 61 |
| | (Forward) | tgg | | | |
| | R-141 | gtatcgtcaa ggcactctt | 4 | 19 | 61 |
| | (Reverse) | | | | |
| C | F-95 | aatgactgaa tataaacttg | 2 | 23 | 61 |
| | (Forward) | tgg | | | |
| | R-147 | tagctgtatc gtcaaggc | 5 | 18 | 61 |
| | (Reverse) | | | | |
| D | F-103 | aatataaact tgtggtagtt | 3 | 23 | 61 |
| | (Forward) | gga | | | |
| | R-141 | gtatcgtcaa ggcactctt | 4 | 19 | 61 |
| | (Reverse) | | | | |

TABLE 3
Sequences (wild-type) amplified
by primer sets A-D

| Prim- | | SEQ |
|---|---|---|
| er | | ID |
| Set | Sequence | NO: |
| A | gaatataaac ttgtggtagt tggagctggt ggcgtaggca | 6 |
| | agagtgcctt gacgatac | |
| B | aatgactgaa tataaacttg tggtagttgg | 7 |
| | agctggtggc gtaggcaaga gtgccttgac | |
| | gatac | |
| C | aatgactgaa tataaacttg tggtagttgg | 8 |
| | agctggtggc gtaggcaaga gtgccttgac | |
| | gatacagcta | |
| D | aatataaact tgtggtagtt ggagctggtg gcgtaggcaa | 9 |
| | gagtgccttg acgatac | |

TABLE 4
Examples of mutations in codons 12 and 13 correlated with cancer and tumors

| Condition | Mutation* | References | Notes |
|---|---|---|---|
| Lung cancer | Gly12Cys (ggt->tgt) | (Nakano et al., 1984); | Gly12Cys first identified in a cell line of human lung cancer; |
| | Gly12Val (ggt->gtt) | (Ahrendt et al., 2001) | K-RAS mutations more common in primary lung |
| | Gly12Ser (ggt->agt) | | adenocarcinomas from smokers, Gly12Cys most common |
| | Gly12Asp (ggt->gat) | | (Ahrendt et al., 2001) |
| | Gly13Asp (ggc->gac) | | |
| Bladder cancer | Gly12Arg (ggt->cgt) | (Santos et at., 1984) | Found in cell lines. |
| Breast | Gly13Asp (ggc->gac) | (Kozma et al., 1987) | Used 3T3 cells transfected with genomic DNA from the |
| adenocarcinoma | | | human breast carcinoma cell line MDA-MB231. |
| Pancreatic | Gly12Asp (ggt->gat) | (Motojima et al., 1993) | Motojima et al. found Gly12Asp most common; followed by |
| carcinoma | Gly12Val (ggt->gtt) | | Gly12Val. |
| | Gly12Arg (ggt->cgt) | | |
| | Gly12Cys (ggt->tgt) | | |
| Pilocytic | Gly13Arg (ggc->cgc) | (Sharma et al., 2005) | |
| astrocytoma | | | |

*Presented as non-limiting examples.

TABLE 5
Examples of amplified sequences of
selected mutations using Set A

| | | SEQ |
|---|---|---|
| | | ID |
| Mutation* | Amplified sequence | NO |
| Gly12Cys | gaatataaac ttgtggtagt | 10 |
| (ggt->tgt) | tggagcttgt ggcgtaggca | |
| | agagtgcctt gacgatac | |
| Gly12Val | gaatataaac ttgtggtagt | 11 |
| (ggt->gtt) | tggagctgtt ggcgtaggca | |
| | agagtgcctt gacgatac | |
| Gly12Ser | gaatataaac ttgtggtagt | 12 |
| (ggt->agt) | tggagctagt ggcgtaggca | |
| | agagtgcctt gacgatac | |
| Glyl2Asp | gaatataaac ttgtggtagt | 13 |
| (ggt->gat) | tggagctgat ggcgtaggca | |
| | agagtgcctt gacgatac | |
| Gly13Asp | gaatataaac ttgtggtagt | 14 |
| (ggc->gac) | tggagctggt gacgtaggca | |
| | agagtgcctt gacgatac | |
| Gly12Arg | gaatataaac ttgtggtagt | 15 |
| (ggt->cgt) | tggagctcgt ggcgtaggca | |
| | agagtgcctt gacgatac | |
| Glyl3Arg | gaatataaac ttgtggtagt | 16 |
| (ggc->cgc) | tggagctggt cgcgtaggca | |
| | agagtgcctt gacgatac | |

TABLE 6
Examples of amplified sequences of
selected mutations using Set B

| | | SEQ |
|---|---|---|
| | | ID |
| Mutation* | Amplified sequence | NO |
| Gly12Cys | aatgactgaa tataaacttg | 17 |
| (ggt->tgt) | tggtagttgg agcttgtggc | |
| | gtaggcaaga gtgccttgac | |
| | gatac | |
| Gly12Val | aatgactgaa tataaacttg | 18 |
| (ggt->gtt) | tggtagttgg agctgttggc | |
| | gtaggcaaga gtgccttgac | |
| | gatac | |
| Gly12Ser | aatgactgaa tataaacttg | 19 |
| (ggt->agt) | tggtagttgg agctagtggc | |
| | gtaggcaaga gtgccttgac | |
| | gatac | |
| Gly12Asp | aatgactgaa tataaacttg | 20 |
| (ggt->gat) | tggtagttgg agctgatggc | |
| | gtaggcaaga gtgccttgac | |
| | gatac | |
| Gly13Asp | aatgactgaa tataaacttg | 21 |
| (ggc->gac) | tggtagttgg agctggtgac | |
| | gtaggcaaga gtgccttgac | |
| | gatac | |
| Gly12Arg | aatgactgaa tataaacttg | 22 |
| (ggt->cgt) | tggtagttgg agctcgtggc | |
| | gtaggcaaga gtgccttgac | |
| | gatac | |
| Gly13Arg | aatgactgaa tataaacttg | 23 |
| (ggc->cgc) | tggtagttgg agctggtcgc | |
| | gtaggcaaga gtgccttgac | |
| | gatac | |

TABLE 7
Examples of amplified sequences of
selected mutations using Set C

| | | SEQ |
|---|---|---|
| | | ID |
| Mutation* | Amplified sequence | NO |
| Gly12Cys | aatgactgaa tataaacttg | 24 |
| (ggt->tgt) | tggtagttgg agcttgtggc | |
| | gtaggcaaga gtgccttgac | |
| | gatacagcta | |
| Gly12Val | aatgactgaa tataaacttg | 25 |
| (ggt->gtt) | tggtagttgg agctgttggc | |
| | gtaggcaaga gtgccttgac | |
| | gatacagcta | |
| Gly12Ser | aatgactgaa tataaacttg | 26 |
| (ggt->agt) | tggtagttgg agctagtggc | |
| | gtaggcaaga gtgccttgac | |
| | gatacagcta | |
| Gly12Asp | aatgactgaa tataaacttg | 27 |
| (ggt->gat) | tggtagttgg agctgatggc | |
| | gtaggcaaga gtgccttgac | |
| | gatacagcta | |
| Gly13Asp | aatgactgaa tataaacttg | 28 |
| (ggc->gac) | tggtagttgg agctggtgac | |
| | gtaggcaaga gtgccttgac | |
| | gatacagcta | |
| Gly12Arg | aatgactgaa tataaacttg | 29 |
| (ggt->cgt) | tggtagttgg agctcgtggc | |
| | gtaggcaaga gtgccttgac | |
| | gatacagcta | |
| Gly13Arg | aatgactgaa tataaacttg | 30 |
| (ggc->cgc) | tggtagttgg agctggtcgc | |
| | gtaggcaaga gtgccttgac | |
| | gatacagcta | |

TABLE 8
Examples of amplified sequences of
selected mutations using Set D

| | | SEQ |
|---|---|---|
| | | ID |
| Mutation* | Amplified sequence | NO |
| Gly12Cys | aatataaact tgtggtagtt | 31 |
| (ggt->tgt) | ggagcttgtg gcgtaggcaa | |
| | gagtgccttg acgatac | |
| Gly12Va1 | aatataaact tgtggtagtt | 32 |
| (ggt->gtt) | ggagctgttg gcgtaggcaa | |
| | gagtgccttg acgatac | |
| Gly12Ser | aatataaact tgtggtagtt | 33 |
| (ggt->agt) | ggagctagtg gcgtaggcaa | |
| | gagtgccttg acgatac | |
| Gly12Asp | aatataaact tgtggtagtt | 34 |
| (ggt->gat) | ggagctgatg gcgtaggcaa | |
| | gagtgccttg acgatac | |
| Gly13Asp | aatataaact tgtggtagtt | 35 |
| (ggc->gac) | ggagctggtg acgtaggcaa | |
| | gagtgccttg acgatac | |
| Gly12Arg | aatataaact tgtggtagtt | 36 |
| (ggt->cgt) | ggagctcgtg gcgtaggcaa | |
| | gagtgccttg acgatac | |
| Gly13Arg | aatataaact tgtggtagtt | 37 |
| (ggc->cgc) | ggagctggtc gcgtaggcaa | |
| | gagtgccttg acgatac | |

In another embodiment, the primer sets are subjected to amplification conditions, wherein the first cycle comprises incubating the reaction mix with at least one nucleic acid polymerase, such as a DNA polymerase, at 94° C. for 10 seconds, followed by 55-60° C. for 20 seconds, and then 72° C. for 20 seconds. The cycle can be repeated multiple times, such as for 40 cycles. A final cycle can be added, wherein the reaction mix is held at 40° C. These conditions can be adjusted as necessary, and easily, by one of skill in the art. Amplification conditions are discussed further below.

After amplification, the reaction mix is subjected to spectrometric analysis, such as ESI-MS. The sample is injected into a spectrometer, the molecular mass or corresponding “base composition signature” (BCS) of any amplification product is then determined and matched against a database of molecular masses or BCS's. A BCS is the exact base composition determined from the molecular mass of a bioagent identifying amplicon. BCS's provide a useful index of nucleic acids. A BCS differs from a nucleic acid sequence in that the signature does not order the bases, but instead represents the nucleic acid base composition of the nucleic acid (e.g., A, G, C, T). The present method thus provides rapid throughput and does not require nucleic acid sequencing of the amplified target sequence for detection and identification. Furthermore, time-consuming separation technologies, such as gel electrophoresis, coupled with detection of the separated sequences, whether from simple gel staining or hybridization with a probe comprising a detectable label, is avoided. In the methods of the invention, mutations in codons 12 and 13 of K-RAS can be detected in a sample with a simple detection step and database interrogation.

In one embodiment, samples are obtained from a subject, which can be a mammal, such as a human. The sample is typically taken from a tumor, or a tissue suspected of harboring a tumor or cancer cells.

Definitions

“Specifically hybridize” refers to the ability of a nucleic acid to bind detectably and specifically to a second nucleic acid. Polynucleotides specifically hybridize with target nucleic acid strands under hybridization and wash conditions that minimize appreciable amounts of detectable binding by non-specific nucleic acids.

“Target sequence” or “target nucleic acid sequence” means a nucleic acid sequence encompassing codon 12 or 13 of a K-RAS gene, or complements thereof, that is amplified, detected, or both using one or more of the polynucleotide primer sets A, B, C, or D. Additionally, while the term target sequence sometimes refers to a double stranded nucleic acid sequence; a target sequence can also be single-stranded. In cases where the target is double-stranded, polynucleotide primer sequences of the present invention preferably amplify both strands of the target sequence. A target sequence can be selected that is more or less specific for a particular organism. For example, the target sequence can be specific to an entire genus, to more than one genus, to a species or subspecies, serogroup, auxotype, serotype, strain, isolate or other subset of organisms.

“Test sample” means a sample taken from a subject, or a biological fluid, wherein the sample may contain a K-RAS target sequence. A test sample can be taken from any source, for example, tissue, blood, saliva, sputa, mucus, sweat, urine, urethral swabs, cervical swabs, urogenital or anal swabs, conjunctival swabs, ocular lens fluid, cerebral spinal fluid, etc. A test sample can be used (i) directly as obtained from the source; or (ii) following a pre-treatment to modify the character of the sample. Thus, a test sample can be pre-treated by, for example, preparing plasma or serum from blood, disrupting cells or viral particles, preparing liquids from solid materials, diluting viscous fluids, filtering liquids, adding reagents, purifying nucleic acids, etc.

“Subjects” include a mammal, a bird, or a reptile. The subject can be a cow, horse, dog, cat, or a primate. Preferably, the subject is a human. Subjects can be alive or dead.

A “polynucleotide” is a nucleic acid polymer of ribonucleic acid (RNA), deoxyribonucleic acid (DNA), modified RNA or DNA, or RNA or DNA mimetics (such as PNAs), and derivatives thereof, and homologues thereof. Thus, polynucleotides include polymers composed of naturally occurring nucleobases, sugars and covalent inter-nucleoside (backbone) linkages as well as polymers having non-naturally-occurring portions that function similarly. Such modified or substituted nucleic acid polymers are well known in the art and for the purposes of the present invention, are referred to as “analogues.” Oligonucleotides are generally short polynucleotides from about 10 to up to about 160 or 200 nucleotides.

A first polynucleotide having sequence identity with a second polynucleotide means a polynucleotide having at least about 60% nucleic acid sequence identity, more preferably at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleic acid sequence identity with the second polynucleotide.

“Percent (%) nucleic acid sequence identity” with respect to nucleic acid sequences is defined as the percentage of nucleotides in a candidate sequence that are identical with the nucleotides in the sequence of interest, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2. ALIGN or Megalign (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:

% nucleic acid sequence identity=W/Z*100

where

W is the number of nucleotides scored as identical matches by the sequence alignment program's or algorithm's alignment of C and D

and

Z is the total number of nucleotides in D.

When the length of nucleic acid sequence C is not equal to the length of nucleic acid sequence D, the % nucleic acid sequence identity of C to D will not equal the % nucleic acid sequence identity of D to C.

“Consisting essentially of a polynucleotide having a % sequence identity” means that the polynucleotide does not substantially differ in length, but may differ substantially in sequence. Thus, a polynucleotide “A” consisting essentially of a polynucleotide having at least 80% sequence identity to a known sequence “B” of 100 nucleotides means that polynucleotide “A” is about 100 nts long, but up to 20 nts can vary from the “B” sequence. The polynucleotide sequence in question can be longer or shorter due to modification of the termini, such as, for example, the addition of 1-15 nucleotides to produce specific types of probes, primers and other molecular tools, etc., such as the case of when substantially non-identical sequences are added to create intended secondary structures. Such non-identical nucleotides are not considered in the calculation of sequence identity when the sequence is modified by “consisting essentially of.”

The specificity of single stranded DNA to hybridize complementary fragments is determined by the stringency of the reaction conditions. Hybridization stringency increases as the propensity to form DNA duplexes decreases. In nucleic acid hybridization reactions, the stringency can be chosen to favor specific hybridizations (high stringency). Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide, which decrease DNA duplex stability. A common approach is to vary the temperature: higher relative temperatures result in more stringent reaction conditions. Excellent explanation of stringency of hybridization reactions are available in the literature (Ausubel et al., 1987).

Hybridization under “stringent conditions” means hybridization protocols in which nucleotide sequences at least 60% homologous to each other remain hybridized.

Polynucleotides can include other appended groups such as peptides (e.g., for targeting host cell receptors in vivo), or agents facilitating transport across the cell membrane. In addition, oligonucleotides can be modified with hybridization-triggered cleavage agents (van der Krol et al., 1988) or intercalating agents (Zon, 1988). The oligonucleotide can be conjugated to another molecule, e.g., a peptide, a hybridization triggered cross-linking agent, a transport agent, a hybridization-triggered cleavage agent, and the like.

Useful polynucleotide analogues include polymers having modified backbones or non-natural inter-nucleoside linkages. Modified backbones include those retaining a phosphorus atom in the backbone, such as phosphorothioates, chiral phosphorothioates, phosphorodithioates, phosphotriesters, aminoalkylphosphotriesters, methyl and other alkyl phosphonates, as well as those no longer having a phosphorus atom, such as backbones formed by short chain alkyl or cycloalkyl inter-nucleoside linkages, mixed heteroatom and alkyl or cycloalkyl inter-nucleoside linkages, or one or more short chain heteroatomic or heterocyclic inter-nucleoside linkages. Modified nucleic acid polymers (analogues) can contain one or more modified sugar moieties.

Analogs that are RNA or DNA mimetics, in which both the sugar and the inter-nucleoside linkage of the nucleotide units are replaced with novel groups, are also useful. In these mimetics, the base units are maintained for hybridization with the target sequence. An example of such a mimetic, which has been shown to have excellent hybridization properties, is a peptide nucleic acid (PNA) (Buchardt et al., 1992; Nielsen et al., 1991). Another example are locked nucleic acids (LNA), where the 2′ and 4′ glycosidic carbons are linked by a 2′-O-methylene bridge (Karkare and Bhatnagar, 2006).

The realm of nucleotides includes derivatives wherein the nucleic acid molecule has been covalently modified by substitution, chemical, enzymatic, or other appropriate means with a moiety other than a naturally occurring nucleotide.

The polynucleotides of the present invention thus comprise primers that specifically hybridize to target sequences, for example the nucleic acid molecules having any one of the nucleic acid sequences of SEQ ID NOs:1-5, including analogues and/or derivatives of the nucleic acid sequences, and homologs thereof. The polynucleotides of the invention can be used as primers to amplify or detect K-RAS-containing polynucleotides.

The polynucleotides of SEQ ID NOs:1-5 can be prepared by conventional techniques, such as solid-phase synthesis using commercially available equipment, such as that available from Applied Biosystems USA Inc. (Foster City, Calif.; USA), DuPont, (Wilmington, Del.; USA), or Milligen (Bedford, Mass.; USA). Modified polynucleotides, such as phosphorothioates and alkylated derivatives, can also be readily prepared by similar methods known in the art (Fino, 1995; Mattingly, 1995; Ruth, 1990).

Practicing the Invention

The invention includes methods for detecting mutations in codon 12 or 13 of K-RAS nucleic acids wherein a test sample is collected; amplification reagents and K-RAS-specific primers such as those of SEQ ID NOs:1-5 are added; the sample subjected to amplification; the amplified nucleic acid (amplicon), if any, is analyzed using mass spectrometry; and the resulting data used to interrogate a database. The BCS may or may not be determined and used.

Amplification of K-RAS Nucleic Acids

The polynucleotides of SEQ ID NOs:1-5 can be used as primers to amplify K-RAS polynucleotides in a sample. The polynucleotides are used as primers, wherein the primer pairs are SEQ ID NOs:1 and 4 (Set A), SEQ ID NOs:2 and 4 (Set B), SEQ ID NOs:2 and 5 (Set C), and SEQ ID NOs:3 and 4 (Set D).

The amplification method generally comprises (a) a reaction mixture comprising nucleic acid amplification reagents, at least one primer set of the present invention, and a test sample suspected of containing a at least one target sequence; and (b) subjecting the mixture to amplification conditions to generate at least one copy of a nucleic acid sequence complementary to the target sequence if the target sequence is present.

Step (b) of the above method can be repeated any suitable number of times prior to, for example, a detection step; e.g., by thermal cycling the reaction mixture between 10 and 100 times (or more), typically between about 20 and about 60 times, more typically between about 25 and about 45 times.

Nucleic acid amplification reagents include enzymes having polymerase activity, enzyme co-factors, such as magnesium or manganese; salts; nicotinamide adenine dinucleotide (NAD); and deoxynucleotide triphosphates (dNTPs), (dATP, dGTP, dCTP and dTTP or ribonucleoside triphosphates).

Amplification conditions are those that promote annealing and extension of one or more nucleic acid sequences. Such annealing is dependent in a rather predictable manner on several parameters, including temperature, ionic strength, sequence length, complementarity, and G:C content of the sequences. For example, lowering the temperature in the environment of complementary nucleic acid sequences promotes annealing. Typically, diagnostic applications use hybridization temperatures that are about 2° C. to 18° C. (e.g., approximately 10° C.) below the melting temperature, Tₘ. Ionic strength also impacts Tₘ. Typical salt concentrations depend on the nature and valency of the cation but are readily understood by those skilled in the art. Similarly, high G:C content and increased sequence length stabilize duplex formation and increases Tₘ.

Finally, the hybridization temperature is selected close to or at the Tₘ of the primers. Thus, obtaining suitable hybridization conditions for a particular primer set is within the ordinary skill of the PCR arts.

Amplification procedures are well-known in the art and include the polymerase chain reaction (PCR), transcription-mediated amplification (TMA), rolling circle amplification, nucleic acid sequence based amplification (NASBA), ligase chain reaction and strand displacement amplification (SDA). One skilled in the art understands that for use in certain amplification techniques, the primers may need to be modified; for example, SDA primers usually comprise additional nucleotides near the 5′ ends that constitute a recognition site for a restriction endonuclease. For NASBA, the primers can include additional nucleotides near the 5′ end that constitute an RNA polymerase promoter. Polynucleotides thus modified are considered to be within the scope of the present invention.

The present invention includes the use of the polynucleotides of SEQ ID NOs:1-5 in methods to specifically amplify target nucleic acid sequences in a test sample in a single vessel format.

Chemical Modification of Primers

Primers can be chemically modified, for example, to improve the efficiency of hybridization. For example, because variation (due to codon wobble in the 3^{rd} position) in conserved regions among species often occurs in the third position of a DNA triplet, the primers of SEQ ID NOs:1-5 can be modified such that the nucleotide corresponding to this position is a “universal base” that can bind to more than one nucleotide. For example, inosine (I) binds to U, C or A; guanine (G) binds to U or C, and uridine (U) binds to A or G. Other examples of universal bases include nitroindoles such as 5-nitroindole or 3-nitropyrrole (Loakes et al., 1995), the degenerate nucleotides dP or dK (Hill et al.), an acyclic nucleoside analog containing 5-nitroindazole (Van Aerschot et al., 1995) or the purine analog 1-(2-deoxy-β-D-ribofuranosyl)-imidazole-4-carboxamide (Sala et al., 1996).

In another embodiment, to compensate for the somewhat weaker binding by the “wobble” base, the oligonucleotide primers can be designed such that the first and second positions of each triplet are occupied by nucleotide analogs which bind with greater affinity than the unmodified nucleotide. Examples of these analogs include 2,6-diaminopurine, which binds to thymine; propyne T, which binds to adenine; and propyne C and phenoxazines, including G-clamp, which bind to G. Propynylated pyrimidines are described in U.S. Pat. Nos. 5,645,985, 5,830,653 and 5,484,908. Phenoxazines are described in U.S. Pat. Nos. 5,502,177, 5,763,588, and 6,005,096. G-clamps are described in U.S. Pat. Nos. 6,007,992 and 6,028,183.

Controls

Various controls can be instituted in the methods of the invention to assure, for example, that amplification conditions are optimal. An internal standard can be included in the reaction. Such internal standards generally comprise a control target nucleic acid sequence. The internal standard can optionally further include an additional pair of primers. The primary sequence of these control primers can be unrelated to the polynucleotides of the present invention and specific for the control target nucleic acid sequence.

In the context of the present invention, a control target nucleic acid sequence is a nucleic acid sequence that:

(a) can be amplified either by a primer or primer pair being used in a particular reaction or by distinct control primers; and

(b) is detected by mass spectrometric techniques.

Mass Spectrometric Characterization of Amplicons

Mass spectrometry (MS)-based detection and characterizing PCR products has several distinct advantages. MS is intrinsically a parallel detection scheme without the need for radioactive or fluorescent labels, since every amplification product is identified by its molecular mass. Less than femtomole quantities of material are required. An accurate assessment of the molecular mass of a sample can be quickly obtained. Intact molecular ions can be generated from amplification products using one of a variety of ionization techniques to convert the sample to gas phase. These ionization methods include electrospray ionization (ES), matrix-assisted laser desorption ionization (MALDI) and fast atom bombardment (FAB). For example, MALDI of nucleic acids, along with examples of matrices for use in MALDI of nucleic acids, are described in WO 98/54751.

Upon ionization, several peaks are observed from one sample due to the formation of ions with different charges. Averaging the multiple readings of molecular mass obtained from a single mass spectrum affords an estimate of molecular mass of the amplicon. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

Suitable mass detectors for the present invention include Fourier transform ion cyclotron resonance mass spectrometry (FT-ICR-MS), ion trap, quadrupole, magnetic sector, time of flight (TOF), Q-TOF, and triple quadrupole.

In general, useful mass spectrometric techniques that can be used in the present invention include tandem mass spectrometry, infrared multiphoton dissociation and pyrolytic gas chromatography mass spectrometry (PGC-MS).

The accurate measurement of molecular mass for large DNAs is limited by the adduction of cations from the PCR reaction to each strand, resolution of the isotopic peaks from natural abundance ¹³C and ¹⁵N isotopes, and assignment of the charge state for any ion. The cations are removed by in-line dialysis using a flow-through chip that brings the solution containing the PCR products into contact with a solution containing ammonium acetate in the presence of an electric field gradient orthogonal to the flow. The latter two problems can be addressed by operating with a resolving power of >100,000 and by incorporating isotopically-depleted nucleotide triphosphates into the DNA. The resolving power of the instrument is also a consideration. At a resolving power of 10,000, the modeled signal from the [M−14H+]¹⁴⁻ charge state of an 84-mer PCR product is poorly characterized and assignment of the charge state or exact mass is impossible. At a resolving power of 33,000, the peaks from the individual isotopic components are visible. At a resolving power of 100,000, the isotopic peaks are resolved to the baseline and assignment of the charge state for the ion is straightforward. The [¹³C, ¹⁵M-depleted triphosphates are obtained, for example, by growing microorganisms on depleted media and harvesting the nucleotides (Batey et al., 1992).

Tandem mass spectrometry techniques can provide more definitive information pertaining to molecular identity or sequence. Tandem MS involves the coupled use of two or more stages of mass analysis where both the separation and detection steps are based on mass spectrometry. The first stage is used to select an ion or component of a sample from which further structural information is to be obtained. The selected ion is then fragmented using, e.g., blackbody irradiation, infrared multiphoton dissociation, or collisional activation. For example, ions generated by electrospray ionization (ESI) can be fragmented using IR multiphoton dissociation. This activation leads to dissociation of glycosidic bonds and the phosphate backbone, producing two series of fragment ions, called the w-series (having an intact 3′ terminus and a 5′ phosphate following internal cleavage) and the a-Base series (having an intact 5′ terminus and a 3′ furan).

The second stage of mass analysis is then used to detect and measure the mass of these resulting fragments of product ions. Such ion selection followed by fragmentation routines can be performed multiple times so as to essentially completely dissect the molecular sequence of a sample.

PCR amplicons when analyzed by ESI-TOF mass spectrometry give a pair of masses, one for each strand of the double-stranded DNA amplicon. In some cases, the molecular mass of one strand alone provides enough information for unambiguously identification. In other cases, however, determining information from both strands is preferred.

The molecular mass of a single strand can also be consistent with more than on BCS. This can also be true for the complementary strand. These ambiguities are resolved when the added constraint of complementarity is applied. Thus a strand with a BCS of A₂₈T₂₄G₂₉C2₅ is paired with its complement A₂₄T₂₈G₂₅C₂₉. Typically when sets of possible BCS solutions for the two strands of an amplicon are compared, usually only one pair of strands are complements of each other. That pair represents a unique solution for an amplicon's BCS; the other potential solutions are discarded because they are non-complementary.

For example, an amplicon is analyzed by ESI-TOF mass spectrometry that gives two masses: a first mass of 32,889.45 Da for one strand, and a second mass of 33,071.46 Da for the second. Assuming an average mass for the DNA bases are as follows:
A=313.0576 amu
G=328.0526 amu
C=289.0464 amu; and
T=304.0461 amu.
   Each strand has 5 possible solutions, each solution resulting in the measured mass. The calculated possible solutions for the first and second strands are:

| First Strand (32,889.45 Da) | Second strand (33,071.46 Da) |
|---|---|
| A₂₄G₂₇C₂₇T₂₄ | A₂₅G₂₆C₃₀T₂₅ |
| A₂₈G₃₁C₂₇T₂₄ | A₂₄G₂₅C₂₉T₂₈ |
| A₂₆G₃₀C₂₅T₂₅ | A₂₅G₂₅C₃₀T₂₆ |
| A₂₈G₂₉C₂₅T₂₄ | A₂₄G₂₇C₃₁T₂₈ |
| A₂₅G₃₀C₂₆T₂₅ | A₂₄G₂₇C₂₇T₂₄ |

Inspecting these possible solutions, there is only one solution where the first strand is the complement of the second strand when the constraint of complementarity is applied; that is, for every A in the first strand, there is a T in the second; for every G in the first strand, there is a C in the second, and so on. Thus, the only solution is:
First strand: A₂₈G₂₉C₂₅T₂₄
Second strand: T₂₉C₂₉G₂₅A₂₄

Mass-modifying “tags” can also be used. A nucleotide analog or “tag” is incorporated during amplification (e.g., a 5-(trifluoromethyl) deoxythymidine triphosphate) that has a different molecular weight than the unmodified base so as to improve distinction of masses. Such tags are described in, for example, WO97/33000. This further limits the number of possible base compositions consistent with any mass. For example, 5-(trifluoromethyl)deoxythymidine triphosphate can be used in place of dTTP in a separate nucleic acid amplification reaction. Measurement of the mass shift between a conventional amplification product and the tagged product is used to quantitate the number of thymidine nucleotides in each of the single strands. Because the strands are complementary, the number of adenosine nucleotides in each strand is also determined.

In another amplification reaction, the number of G and C residues in each strand is determined using, for example, the cytidine analog 5-methylcytosine (5-meC) or propyne C. The combination of the A/T reaction and G/C reaction, followed by molecular weight determination, provides a unique base composition. This method is summarized in Table 9.

TABLE 9

| | Double strand | Single strand | Total mass | Base info | Base info | Total base | Total base |
|---|---|---|---|---|---|---|---|
| Mass tag | sequence | sequence | (this strand) | (this strand) | (other strand) | comp. (top) | comp. (bottom) |
| T*mass | T*ACGT*ACGT* | T*ACGT*ACGT* | 3x | 3T | 3A | | |
| (T* − T) = x | AT*GCAT*GCA | | | | | | |
| | | AT*GCAT*GCA | 2x | 2T | 2A | 3T | 3A |
| | | | | | | 2A | 2T |
| C*mass | TAC*GTAC*GT | TAC*GTAC*GT | 2y | 2T | 2G | 2C | 2G |
| (C* − C) = y | ATGC*ATGC*A | | | | | 2G | 2C |
| | | ATGC*ATGC*A | 2y | 2C | 2G | | |

In Table 9, the mass tag phosphorothioate A (A*) was used to distinguish a Bacillus anthracis cluster. The B. anthracis (A₁₄G₉C₁₄T₉) had an average MW of 14072.26, and the B. anthracis (A₁A*₁₃G₉C₁₄T₉) had an average molecular weight of 14281.11 and the phosphorothioate A had an average molecular weight of +16.06 as determined by ESI-TOF MS.

In another example, assume the measured molecular masses of each strand are 30,000.115 Da and 31,000.115 Da respectively, and the measured number of dT and dA residues are (30,28) and (28,30). If the molecular mass is accurate to 100 ppm, there are 7 possible combinations of dG+dC possible for each strand. However, if the measured molecular mass is accurate to 10 ppm, there are only 2 combinations of dG+dC, and at 1 ppm accuracy there is only one possible base composition for each strand.

Base Composition Signatures as Indices of Identifying Amplicons and Database Interrogation

Conversion of molecular mass data to a base composition signature is useful for certain analyses. A “base composition signature” (BCS) is the exact base composition determined from the molecular mass of an amplicon. The BCS can provide an index of a specific gene in a specific organism (See, for example, U.S. Pat. Nos. 7,217,510, 7,108,974, 7,255,992 and 7,226,739).

Base compositions, like sequences, vary slightly from isolate to isolate within species. It is possible to manage this diversity by building “base composition probability clouds” around the composition constraints for each K-RAS mutation. This permits identifying a K-RAS mutation in a fashion similar to sequence analysis. A “pseudo four-dimensional plot” can be used to visualize the concept of base composition probability clouds.

The BCS's collected from mass spectrometric analysis can be used to query a database that contains, for example, the information from sequences of known mutations, such as thus shown in Table 1 and known in the literature for codons 12 and 13 of K-RAS, wherein the mutations correlate with the presence of at least one cancer or tumor cell. From this interrogation, the species of K-RAS mutation can be identified from the amplified target sequence.

Databases

The invention in part exploits K-RAS sequences known to be mutated in a large number of cancers and tumors, wherein the polynucleotides of the invention, SEQ ID Nos:1-5, are designed to hybridize to sequences that flank the mutated regions, specifically codons 12 and 13. Databases useful for the invention contain known K-RAS codon 12 and 13 molecular masses and BCS's of the targeted sequences (as defined by the primer sets A, B, C, and D) and, optionally, BCS's and masses from wild-type K-RAS sequences. An example of data that could be compiled into a simple database is shown in Table 10, which shows the BCS's, G+C content and molecular mass, as derived from the amplified sequences from the primer sets A, B, C, and D (resulting in the sequences of SEQ ID Nos:6-37; note that the amplified sequences include the primer sequences themselves).

TABLE 10
Data from Amplified Target Sequences

| | | | | | | | Average |
|---|---|---|---|---|---|---|---|
| Primer | SEQ ID | BCS (%*) | | | | G + C | molecular |
| Set | NO | A | T | C | G | (%) | mass |
| A | 6 (WT**) | 25.9 | 25.9 | 13.8 | 35.5 | 49.3 | 35873.2 |
| | 10 | 25.9 | 27.6 | 13.8 | 32.8 | 46.6 | 35872.2 |
| | 11 | 25.9 | 27.6 | 13.8 | 32.8 | 46.6 | 35872.2 |
| | 12 | 27.6 | 25.9 | 13.8 | 32.8 | 46.6 | 35872.2 |
| | 13 | 27.6 | 25.9 | 13.8 | 32.8 | 46.6 | 35872.2 |
| | 14 | 27.6 | 25.9 | 13.8 | 32.8 | 46.6 | 35872.2 |
| | 15 | 25.9 | 25.9 | 15.5 | 32.8 | 48.3 | 35873.2 |
| | 16 | 25.9 | 25.9 | 15.5 | 32.8 | 48.3 | 35873.2 |
| B | 7 (WT) | 27.7 | 26.2 | 13.8 | 32.3 | 46.1 | 40197.0 |
| | 17 | 27.7 | 27.7 | 13.8 | 30.8 | 44.6 | 40196.0 |
| | 18 | 27.7 | 27.7 | 13.8 | 30.8 | 44.6 | 40196.0 |
| | 19 | 29.2 | 26.2 | 13.8 | 30.8 | 44.6 | 40196.0 |
| | 20 | 29.2 | 26.2 | 13.8 | 30.8 | 44.6 | 40196.0 |
| | 21 | 29.2 | 26.2 | 13.8 | 30.8 | 44.6 | 40196.0 |
| | 22 | 27.7 | 26.2 | 15.4 | 30.8 | 46.2 | 40197.0 |
| | 23 | 27.7 | 26.2 | 15.4 | 30.8 | 46.2 | 40197.0 |
| C | 8 (WT) | 28.6 | 25.7 | 14.3 | 31.4 | 45.7 | 43286.0 |
| | 24 | 28.6 | 27.1 | 14.3 | 30.0 | 44.3 | 43285.0 |
| | 25 | 28.6 | 27.1 | 14.3 | 30.0 | 44.3 | 43285.0 |
| | 26 | 30.0 | 25.7 | 14.3 | 30.0 | 44.3 | 43285.0 |
| | 27 | 30.0 | 25.7 | 14.3 | 30.0 | 44.3 | 43285.0 |
| | 28 | 30.0 | 25.7 | 14.3 | 30.0 | 44.3 | 43285.0 |
| | 29 | 28.6 | 25.7 | 15.7 | 30.0 | 45.7 | 43286.0 |
| | 30 | 28.6 | 25.7 | 15.7 | 30.0 | 45.7 | 43286.0 |
| D | 9 (WT) | 26.3 | 26.3 | 14.0 | 33.3 | 47.0 | 35254.8 |
| | 31 | 26.3 | 28.1 | 14.0 | 34.6 | 45.6 | 35253.8 |
| | 32 | 26.3 | 28.1 | 14.0 | 34.6 | 45.6 | 35253.8 |
| | 33 | 28.1 | 26.3 | 14.0 | 31.6 | 45.6 | 35253.8 |
| | 34 | 28.1 | 26.3 | 14.0 | 31.6 | 45.6 | 35253.8 |
| | 35 | 28.1 | 26.3 | 14.0 | 31.6 | 45.6 | 35253.8 |
| | 36 | 26.3 | 26.3 | 15.8 | 31.6 | 47.4 | 35254.8 |
| | 37 | 26.3 | 26.3 | 15.8 | 31.6 | 47.4 | 35254.8 |

*Rounded to the nearest 0.1.
**WT, wild-type sequence

Kits

The polynucleotides of SEQ ID NOs:1-5 can be included as part of kits that allow for the detection of mutations in codons 12 and 13 in K-RAS nucleic acids. Such kits comprise one or more of the polynucleotides of the invention. In one embodiment, the polynucleotides are provided in the kits in combinations for use as primers to specifically amplify K-RAS nucleic acids in a test sample.

Kits for the detection of K-RAS nucleic acids can also include a control target nucleic acid. Kits can also include control primers, which specifically amplify a sequence of the control target nucleic acid sequence.

Kits can also include amplification reagents, reaction components and/or reaction vessels. One or more of the polynucleotides can be modified as previously discussed. One or more of the components of the kit may be lyophilized, and the kit can further include reagents suitable for reconstituting the lyophilized products. The kit can additionally contain instructions for use.

In an additional embodiment, the kits further contain computer-readable media that contains a database that allows for the identification of BCS's. Optionally, the computer-readable media can contain software that allows for data collection and/or database interrogation. Kits can also be supplied with instructional materials. Instructions may be printed on paper or other substrate, and/or may be supplied as an electronic-readable medium, such as a floppy disc, CD-ROM, DVD-ROM, Zip disc, videotape, audiotape, etc.

When a kit is supplied, the different components of the composition can be packaged in separate containers and admixed immediately before use. Such packaging of the components separately can permit long-term storage of the active components. For example, the polynucleotides; a control substrate; and amplification enzyme are supplied in separate containers.

The reagents included in the kits can be supplied in containers of any sort such that the different components are preserved and are not adsorbed or altered by the materials of the container. For example, sealed glass ampoules can contain one of more of the reagents or buffers that have been packaged under a neutral, non-reacting gas, such as nitrogen. Ampoules can consist of any suitable material, such as glass, organic polymers, such as polycarbonate, polystyrene, etc.; ceramic, metal or any other material typically used to hold similar reagents. Other examples of suitable containers include simple bottles that can be fabricated from similar substances as ampoules, and envelopes, that can have foil-lined interiors, such as aluminum or an alloy. Other containers include test tubes, vials, flasks, bottles, syringes, etc.

### EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1

### Primer Design

In this example, four primer sets suitable for use in polymerase chain reactions and other polynucleotide amplification protocols were designed to produce amplification products that were suitable for mass spectrometric analysis to allow identification of mutations in the human v-Ki-ras2 Kristen rat sarcoma viral oncogene homolog, codons 12 and 13. Primers were designed to flank codons 12 and 13 (both of which are glycine in the wild-type sequence).

The primers of SEQ ID NOs:1-5 were designed using OLIGO 6 software (Molecular Biology Insights, Inc.; Cascade, Colo.), using the following design parameters:

200 nM primer concentration

50 mM monovalent cation

0.7 mM free divalent cation

The primer sets A-D, as shown in Table 2, were identified.

### Example 2

### Demonstration of Primer Efficacy to Amplify Target Sequence (Prophetic)

Four primer sets, A (SEQ ID NOs:1 and 4), B (SEQ ID NOs:2 and 4), C (SEQ ID NOs:2, 5), and D (SEQ ID NOs:3, 4) as shown in Table 1 and reproduced in Table 11, are tested for their ability to amplify the target sequence and for the amplified sequence to be detected. The primers themselves can further incorporate a nucleotide analog, such as inosine, uridine, 2,6-diaminopurine, propyne C or propyne T.

TABLE 11
Primer pair sets and sequences

| | | | SEQ | | |
|---|---|---|---|---|---|
| | | | ID | Length, | Tm, |
| Set | Primer | Sequence | NO | nt | ° C. |
| A | F-102 | gaatataaac ttgtggtagt | 1 | 23 | 61 |
| | (Forward) | tgg | | | |
| | R-141 | gtatcgtcaa ggcactctt | 4 | 19 | 61 |
| | (Reverse) | | | | |
| B | F-95 | aatgactgaa tataaacttg | 2 | 23 | 61 |
| | (Forward) | tgg | | | |
| | R-141 | gtatcgtcaa ggcactctt | 4 | 19 | 61 |
| | (Reverse) | | | | |
| C | F-95 | aatgactgaa tataaacttg | 2 | 23 | 61 |
| | (Forward) | tgg | | | |
| | R-147 | tagctgtatc gtcaaggc | 5 | 18 | 61 |
| | (Reverse) | | | | |
| D | F-103 | aatataaact tgtggtagtt | 3 | 23 | 61 |
| | (Forward) | gga | | | |
| | R-141 | gtatcgtcaa ggcactctt | 4 | 19 | 61 |
| | (Reverse) | | | | |

The conditions for the PCR are:

| | | | |
|---|---|---|---|
| 94° | C. | 1 × 10 sec. | 40 cycles |
| 55-60° | C. | 1 × 20 sec. | |
| 72° | C. | 1 × 20 sec. | |
| 4° | C. | hold | 1 cycle |

Alternatively, a “hot start” polymerase can be used in order to avoid false priming during the initial rounds of PCR. Adjustments to the PCR cycling parameters include a heat activation step prior to the standard PCR cycling:

| | | | | |
|---|---|---|---|---|
| 94° | C. | 5-10 | min. | 1 cycle |
| 94° | C. | 1 × 10 | sec. | 40 cycles |
| 55-60° | C. | 1 × 20 | sec. | |
| 72° | C. | 1 × 20 | sec. | |

| | | | |
|---|---|---|---|
| 4° | C. | hold | 1 cycle |

The predicted amplification products are subjected to mass spectrometric analysis, their BCS's determined and coupled with database interrogation, wherein the database contains mutation information from mutations in codons 12 and 13 of K-RAS, including the mass and/or BCS of each target sequence.

### Example 3

### Mass Spectrometry (Prophetic)

Fourier transform ion cyclotron resonance (FTICR) mass spectrometry Instrumentation: The FT-ICR instrument is based on a 7 tesla actively shielded superconducting magnet and modified Bruker Daltonics Apex II 70e ion optics (Bruker Daltonics; Billerica; Mass.) and vacuum chamber. The spectrometer is interfaced to a LEAP PAL autosampler (LEAP Technologies; Carrboro, N.C.) and a custom fluidics control system for high throughput screening applications. Samples are analyzed directly from 96-well or 384-well microtiter plates at a rate of about 1 sample/minute. The Bruker data-acquisition platform is supplemented with a lab-built ancillary data station that controls the autosampler and contains an arbitrary waveform generator capable of generating complex rf-excite waveforms (frequency sweeps, filtered noise, stored waveform inverse Fourier transform (SWIFT), etc.) for tandem MS experiments. Typical performance characteristics include mass resolving power in excess of 100,000 (FWHM), low ppm mass measurement errors, and an operable m/z range between 50 and 5000 m/z.

Modified ESI Source: In sample-limited analyses, analyte solutions are delivered at 150 nL/minute to a 30 mm i.d. fused-silica ESI emitter mounted on a 3-D micromanipulator. The ESI ion optics consists of a heated metal capillary, an rf-only hexapole, a skimmer cone, and an auxiliary gate electrode. The 6.2 cm rf-only hexapole is comprised of 1 mm diameter rods and is operated at a voltage of 380 Vpp at a frequency of 5 MHz. An electro-mechanical shutter can be used to prevent the electrospray plume from entering the inlet capillary unless triggered to the “open” position via a TTL pulse from the data station. When in the “closed” position, a stable electrospray plume is maintained between the ESI emitter and the face of the shutter. The back face of the shutter arm contains an elastomeric seal that can be positioned to form a vacuum seal with the inlet capillary. When the seal is removed, a 1 mm gap between the shutter blade and the capillary inlet allows constant pressure in the external ion reservoir regardless of whether the shutter is in the open or closed position. When the shutter is triggered, a “time slice” of ions is allowed to enter the inlet capillary and is subsequently accumulated in the external ion reservoir. The rapid response time of the ion shutter (<25 ms) provides reproducible, user defined intervals during which ions can be injected into and accumulated in the external ion reservoir.

Apparatus for Infrared Multiphoton Dissociation: A 25-watt CW CO₂ laser operating at 10.6 μm is interfaced to the spectrometer to enable infrared multiphoton dissociation (IRMPD) for tandem MS applications. An aluminum optical bench is positioned approximately 1.5 m from the actively shielded superconducting magnet such that the laser beam is aligned with the central axis of the magnet. Using standard infrared-compatible mirrors and kinematic mirror mounts, the unfocused 3 mm laser beam is aligned to traverse directly through the 3.5 mm holes in the trapping electrodes of the FT-ICR trapped ion cell and longitudinally traverse the hexapole region of the external ion guide finally impinging on the skimmer cone. This scheme allows infrared multiphoton dissociation (IRMPD) to be conducted in an m/z selective manner in the trapped ion cell (e.g. following a SWIFT isolation of the species of interest), or in a broadband mode in the high pressure region of the external ion reservoir where collisions with neutral molecules stabilize IRMPD-generated metastable fragment ions resulting in increased fragment ion yield and sequence coverage.

### Example 4

### Assaying for the Presence of Codon 12 and 13 K-RAS Mutations from a Test Sample (Prophetic)

A sample from an organism or subject suspected of carrying a mutation in human K-RAS, codon 12 and/or 13 is processed using well-known methods and is assayed using primer set A, B, C and/or D using PCR using standard methods, such as those shown in Example 2. The amplified products are assayed by mass spectrometry using the set-up described in Example 3.

If necessary, nucleic acid is isolated from the samples, for example, by cell lysis, centrifugation and ethanol precipitation or any other technique well known in the art.

Mass measurement accuracy can be assayed using an internal mass standard in the ESI-MS study of PCR products. A mass standard, such as a 20-mer phosphorothioate oligonucleotide added to a solution containing a primer set A, B, C and/or D PCR product(s) can be used.

The PCR products are subjected to a step to remove PCR reactants and any confounding metal cations, The PCR products are anchored to a solid support, washed 1-5 times, and then the PCR products eluted from the solid support.

The amplification products are subjected to mass spectrometric analysis coupled with database interrogation, wherein the database contains the information from the regions targeted by the primer sets A (SEQ ID NOs:1, 4), B (SEQ ID NOs:2, 4), C (SEQ ID NOs:2, 5), and D (SEQ ID NOs:3, 4) including the mass and/or BCS of each target sequence (such as the data provided in Table 9).

### REFERENCES

Aaserud, D. J., N. L. Kelleher, D. P. Little, et al. 1996. Accurate base composition of double-strand DNA by mass spectrometry. J. Am. Soc. Mass Spec. 7:1266-1269.
Ahrendt, S. A., P. A. Decker, E. A. Alawi, et al. 2001. Cigarette smoking is strongly associated with mutation of the K-ras gene in patients with primary adenocarcinoma of the lung. Cancer. 92:1525-30.
Almoguera, C., D. Shibata, K. Forrester, et al. 1988. Most human carcinomas of the exocrine pancreas contain mutant c-K-ras genes. Cell. 53:549-54.
Andreyev, H. J., A. R. Norman, D. Cunningham, et al. 2001. Kirsten ras mutations in patients with colorectal cancer: the ‘RASCAL II’ study. Br J Cancer. 85:692-6.
Ausubel, F. M., R. Brent, R. E. Kingston, et al. 1987. Current protocols in molecular biology. John Wiley & Sons, New York.
Barbacid, M. 1987. ras genes. Annu Rev Biochem. 56:779-827.
Batey, R. T., M. Inada, E. Kujawinski, et al. 1992. Preparation of isotopically labeled ribonucleotides for multidimensional NMR spectroscopy of RNA. Nucleic Acids Res. 20:4515-23.
Bezieau, S., M. C. Devilder, H. Avet-Loiseau, et al. 2001. High incidence of N and K-Ras activating mutations in multiple myeloma and primary plasma cell leukemia at diagnosis. Hum Mutat. 18:212-24.
Bos, J. L. 1989. ras oncogenes in human cancer: a review. Cancer Res. 49:4682-9.
Brink, M., A. F. de Goeij, M. P. Weijenberg, et al. 2003. K-ras oncogene mutations in sporadic colorectal cancer in The Netherlands Cohort Study. Carcinogenesis. 24:703-10.
Buchardt, O, P. Nielsen, and R. Berg. 1992. PEPTIDE NUCLEIC ACIDS.
Burmer, G. C., and L. A. Loeb. 1989. Mutations in the KRAS2 oncogene during progressive stages of human colon carcinoma. Proc Natl Acad Sci USA. 86:2403-7.
Ellis, R. W., D. Defeo, T. Y. Shih, et al. 1981. The p21 src genes of Harvey and Kirsten sarcoma viruses originate from divergent members of a family of normal vertebrate genes. Nature. 292:506-11.
Fino, J. U.S. Pat. No. 5,464,746. 1995. HAPTENS, TRACERS, IMMUNOGENS AND ANTIBODIES FOR CARBAZOLE AND DIBENZOFURAN DERIVATIVES.
Grimmond, S. M., D. Raghavan, and P. J. Russell. 1992. Detection of a rare point mutation in Ki-ras of a human bladder cancer xenograft by polymerase chain reaction and direct sequencing. Urol Res. 20:121-6.
Hruban, R. H., A. D. van Mansfeld, G. J. Offerhaus, et al. 1993. K-ras oncogene activation in adenocarcinoma of the human pancreas. A study of 82 carcinomas using a combination of mutant-enriched polymerase chain reaction analysis and allele-specific oligonucleotide hybridization. Am J Pathol. 143:545-54.
Hurst, G. B., M. J. Doktycz, A. A. Vass, et al. 1996. Detection of bacterial DNA polymerase chain reaction products by matrix-assisted laser desorption/ionization mass spectrometry. Rapid Commun Mass Spectrom. 10:377-82.
Karkare, S., and D. Bhatnagar. 2006. Promising nucleic acid analogs and mimics: characteristic features and applications of PNA, LNA, and morpholino. Appl Microbiol Biotechnol. 71:575-86.
Kozma, S. C., M. E. Bogaard, K. Buser, et al. 1987. The human c-Kirsten ras gene is activated by a novel mutation in codon 13 in the breast carcinoma cell line MDA-MB231. Nucleic Acids Res. 15:5963-71.
Kranenburg, O. 2005. The KRAS oncogene: past, present, and future. Biochim Biophys Acta. 1756:81-2.
Laghi, L., O. Orbetegli, P. Bianchi, et al. 2002. Common occurrence of multiple K-RAS mutations in pancreatic cancers with associated precursor lesions and in biliary cancers. Oncogene. 21:4301-6.
Lee, K. H., J. S. Lee, C. Suh, et al. 1995. Clinicopathologic significance of the K-ras gene codon 12 point mutation in stomach cancer. An analysis of 140 cases. Cancer. 75:2794-801.
Liu, E., B. Hjelle, R. Morgan, et al. 1987. Mutations of the Kirsten-ras proto-oncogene in human preleukaemia. Nature. 330:186-8.
Loakes, D., F. Hill, S. Linde, et al. 1995. Nitroindoles as universal bases. Nucleosides Nucleotides. 14:1001-1003.
Mattingly, P. U.S. Pat. No. 5,424,414. 1995. HAPTENS, TRACERS, IMMUNOGENS AND ANTIBODIES FOR 3-PHENYL-A-ADAMANTANEACETIC ACIDS.
Motojima, K., T. Urano, Y. Nagata, et al. 1993. Detection of point mutations in the Kirsten-ras oncogene provides evidence for the multicentricity of pancreatic carcinoma. Ann Surg. 217:138-43.
Muddiman, D., and R. Smith. 1998. Sequencing and Characterization of Larger Oligonucleotides by Electrospray Ionization Fourier Transform Ion Cyclotron Resonance Mass Spectrometry. Rev. Anal. Chem., 17:1-68.
Muddiman, D. C., G. A. Anderson, S. A. Hofstadler, et al. 1997. Length and base composition of PCR-amplified nucleic acids using mass measurements from electrospray ionization mass spectrometry. Anal Chem. 69:1543-9.
Muddiman, D. C., A. P. Null, and J. C. Hannis. 1999. Precise mass measurement of a double-stranded 500 base-pair (309 kDa) polymerase chain reaction product by negative ion electrospray ionization Fourier transform ion cyclotron resonance mass spectrometry. Rapid Commun. Mass Spectrom. 13:1201-1204.
Nakano, H., F. Yamamoto, C. Neville, et al. 1984. Isolation of transforming sequences of two human lung carcinomas: structural and functional analysis of the activated c-K-ras oncogenes. Proc Natl Acad Sci USA. 81:71-5.
Nielsen, P. E., M. Egholm, R. H. Berg, et al. 1991. Sequence-selective recognition of DNA by strand displacement with a thymine-substituted polyamide. Science. 254:1497-500.
Nikiforova, M. N., R. A. Lynch, P. W. Biddinger, et al. 2003. RAS point mutations and PAX8-PPAR gamma rearrangement in thyroid tumors: evidence for distinct molecular pathways in thyroid follicular carcinoma. J Clin Endocrinol Metab. 88:2318-26.
Otori, K., Y. Oda, K. Sugiyama, et al. 1997. High frequency of K-ras mutations in human colorectal hyperplastic polyps. Gut. 40:660-3.
Papageorge, A., D. Lowy, and E. M. Scolnick. 1982. Comparative biochemical properties of p21 ras molecules coded for by viral and cellular ras genes. J Virol. 44:509-19.
Rodenhuis, S., M. L. van de Wetering, W. J. Mooi, et al. 1987. Mutational activation of the K-ras oncogene. A possible pathogenetic factor in adenocarcinoma of the lung. N Engl J Med. 317:929-35.
Ruth, J. U.S. Pat. No. 4,948,882. 1990. Ruth, J. 1990. SINGLE-STRANDED LABELELED OLIGONUCLEOTIDES, REACTIVE MONOMERS AND METHODS OF SYNTHESIS.
Sala, M., V. Pezo, S. Pochet, et al. 1996. Ambiguous base pairing of the purine analogue 1-(2-deoxy-beta-D-ribofuranosyl)-imidazole-4-carboxamide during PCR. Nucleic Acids Res. 24:3302-6.
Samowitz, W. S., K. Curtin, D. Schaffer, et al. 2000. Relationship of Ki-ras mutations in colon cancers to tumor location, stage, and survival: a population-based study. Cancer Epidemiol Biomarkers Prev. 9:1193-7.
Santos, E., D. Martin-Zanca, E. P. Reddy, et al. 1984. Malignant activation of a K-ras oncogene in lung carcinoma but not in normal tissue of the same patient. Science. 223:661-4.
Sharma, M. K., B. A. Zehnbauer, M. A. Watson, et al. 2005. RAS pathway activation and an oncogenic RAS mutation in sporadic pilocytic astrocytoma. Neurology. 65:1335-6.
Shimizu, K., M. Goldfarb, Y. Suard, et al. 1983. Three human transforming genes are related to the viral ras oncogenes. Proc Natl Acad Sci USA. 80:2112-6.
Smit, V. T., A. J. Boot, A. M. Smits, et al. 1988. KRAS codon 12 mutations occur very frequently in pancreatic adenocarcinomas. Nucleic Acids Res. 16:7773-82.
Van Aerschot, A., C. Hendrix, G. Schepers, et al. 1995. In search of acyclic analogs as universal nucleosides in degenerate probes. Nucleosides Mucleotides. 14:1053-1056.
van der Krol, A. R., J. N. Mol, and A. R. Stuitje. 1988. Modulation of eukaryotic gene expression by complementary RNA or DNA sequences. Biotechniques. 6:958-76.
Wunschel, D. S., D. C. Muddiman, K. F. Fox, et al. 1998. Heterogeneity in Bacillus cereus PCR products detected by ESI-FTICR mass spectrometry. Anal Chem. 70:1203-7.
Yanez, L., J. Groffen, and D. M. Valenzuela. 1987. c-K-ras mutations in human carcinomas occur preferentially in codon 12. Oncogene. 1:315-8.
Zenker, M., K. Lehmann, A. L. Schulz, et al. 2007. Expansion of the genotypic and phenotypic spectrum in patients with KRAS germline mutations. J Med Genet. 44:131-5.
Zon, G. 1988. Oligonucleotide analogues as potential chemotherapeutic agents. Pharm Res. 5:539-49.

1. A method of identifying the presence or absence of a mutation in codon 12 or 13 of human K-RAS in a test sample, comprising:
providing a test sample;
forming a reaction mixture comprising:
a primer pair set selected from the group consisting of set A, B, C, and D, wherein:
set A comprises a forward primer comprising a nucleic acid sequence of SEQ ID
NO:1, and a reverse primer comprising a nucleic acid sequence of SEQ ID NO:4; set B comprises a forward primer comprising a nucleic acid sequence of SEQ ID
NO:2, and a reverse primer comprising a nucleic acid sequence of SEQ ID NO:4; set C comprises a forward primer comprising a nucleic acid sequence of SEQ ID
NO:2, and a reverse primer comprising a nucleic acid sequence of SEQ ID NO:5; and set D comprises a forward primer comprising a nucleic acid sequence of SEQ ID
NO:3, and a reverse primer comprising a nucleic acid sequence of SEQ ID NO:4;
subjecting the mixture to amplification conditions to generate an amplification product;
determining the molecular mass of the amplification product; and
comparing the molecular mass of the amplification product to calculated or measured molecular masses of target sequences in a database to identify the presence or absence of a mutation in codon 12 or 13 of human K-RAS.

2. A method of identifying the presence or absence of a mutation in codon 12 or 13 of human K-RAS in a test sample, comprising:
providing a test sample;
forming a reaction mixture comprising:
a primer pair set selected from the group consisting of set A, B, C, and D, wherein:
set A comprises a forward primer comprising a nucleic acid sequence of SEQ ID NO:1, and a reverse primer comprising a nucleic acid sequence of SEQ ID NO:4;
set B comprises a forward primer comprising a nucleic acid sequence of SEQ ID NO:2, and a reverse primer comprising a nucleic acid sequence of SEQ ID NO:4;
set C comprises a forward primer comprising a nucleic acid sequence of SEQ ID NO:2, and a reverse primer comprising a nucleic acid sequence of SEQ ID NO:5;
set D comprises a forward primer comprising a nucleic acid sequence of SEQ ID NO:3, and a reverse primer comprising a nucleic acid sequence of SEQ ID NO:4;
subjecting the mixture to amplification conditions to generate an amplification product;
determining the base composition of the amplification product; and
comparing the base composition of the amplification product to calculated or measured base compositions of target sequences in a database to identify the presence or absence of a mutation in codon 12 or 13 of human K-RAS.

3. The method of claim 1, wherein the identifying the target sequence does not comprise sequencing the amplification product.

4. The method of claim 1, wherein the mass spectrometry is Fourier transform ion cyclotron resonance mass spectrometry (FT-ICR-MS), time of flight mass spectrometry (TOF-MS), or electrospray ionization time of flight spectroscopy.

5. The method of claim 1, wherein the primer set comprises at least one nucleotide analog.

6. The method of claim 5, wherein the nucleotide analog is selected from the group consisting of inosine, uridine, 2,6-diaminopurine, propyne C, and propyne T.

7. The method of claim 1 erg, wherein a molecular mass-modifying tag is incorporated into the amplification product.

8. The method of claim 1, wherein the mutation in codon 12 or 13 of human K-RAS correlates with a cancer selected from the group consisting of colorectal, non-small cell lung, ovarian, bile duct, pancreatic, esophageal, breast, thyroid, endometrial and any other cancer or tumor which presence correlates with a mutation in codon 12 or 13.

9. The method of claim 1, further comprising a step of removing PCR reactants and cations before the step of determining the molecular mass of the amplification product.

10. A method of identifying the presence or absence of a mutation in codon 12 or 13 of human K-RAS in a test sample, comprising:
providing a test sample;
forming a reaction mixture comprising:
a primer pair set selected from the group consisting of set A, B, C, and D, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:4
set B comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:4;
set C comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:5; and
set D comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4,
subjecting the mixture to amplification conditions to generate an amplification product;
determining the molecular mass of the amplification product; and
comparing the molecular mass of the amplification product to calculated or measured molecular masses of target sequences in a database to identify the presence or absence of a mutation in codon 12 or 13 of human K-RAS.

11. A method of identifying the presence or absence of a mutation in codon 12 or 13 of human K-RAS in a test sample, comprising:
providing a test sample;
forming a reaction mixture comprising:
a primer pair set selected from the group consisting of set A, B, C, and D, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:4;
set B comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:4;
set C comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:5; and
set D comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4;
subjecting the mixture to amplification conditions to generate an amplification product;
determining the base composition of the amplification product; and
comparing the base composition of the amplification product to calculated or measured base compositions of target sequences in a database to identify the presence or absence of a mutation in codon 12 or 13 of human K-RAS,

12. The method of claim 10, wherein the identifying the target sequence does not comprise sequencing the amplification product.

13. The method of claim 10, wherein the mass spectrometry is Fourier transform ion cyclotron resonance mass spectrometry (FT-ICR-MS), time of flight mass spectrometry (TOF-MS), or electrospray ionization time of flight spectroscopy.

14. The method of claim 10, wherein the primer set comprises at least one nucleotide analog.

15. The method of claim 14, wherein the nucleotide analog is selected from the group consisting of inosine, uridine, 2,6-diaminopurine, propyne C, and propyne T.

16. The method of claim 10, wherein a molecular mass-modifying tag is incorporated into the amplification product.

17. The method of claim 10, wherein the mutation in codon 12 or 13 of human K-RAS correlates with a cancer selected from the group consisting of colorectal, non-small cell lung, ovarian, bile duct, pancreatic, esophageal, breast, thyroid, endometrial, and any other cancer or tumor which presence correlates with a mutation in codon 12 or 13.

18. A kit, comprising a primer pair set selected from the group consisting of set A, B, C and D, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:4
set B comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:4;
set C comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:5; and
set D comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4;
and amplification reagents.

19. A kit, comprising a primer pair set selected from the group consisting of set A, B, C, and D, wherein:
set A comprises a forward primer comprising a nucleic acid sequence of SEQ ID NO:1, and a reverse primer comprising a nucleic acid sequence of SEQ ID NO:4;
set B comprises a forward primer comprising a nucleic acid sequence of SEQ ID NO:2, and a reverse primer comprising a nucleic acid sequence of SEQ ID NO:4;
set C comprises a forward primer comprising a nucleic acid sequence of SEQ ID NO:2, and a reverse primer comprising a nucleic acid sequence of SEQ ID NO:5; and
set D comprises a forward primer comprising a nucleic acid sequence of SEQ ID NO:3, and a reverse primer comprising a nucleic acid sequence of SEQ ID NO:4; and amplification reagents.

20. A primer pair set selected from the group consisting of A, B, C, and D, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:4
set B comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:4;
set C comprises a forward primer comprising a nucleic acid sequence having at least 80% sequence identity with a nucleic acid sequence of SEQ ID NO:2, and a reverse primer comprising a nucleic acid sequence having at least 80% sequence identity with a nucleic acid sequence of SEQ ID NO:5; and
set D comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4.

21. A primer pair set selected from the group consisting of A, B, C, and D, wherein:
set A comprises a forward primer comprising a nucleic acid sequence of SEQ ID NO:1, and a reverse primer comprising a nucleic acid sequence of SEQ ID NO:4;
set B comprises a forward primer comprising a nucleic acid sequence of SEQ ID NO:2, and a reverse primer comprising a nucleic acid sequence of SEQ ID NO:4;
set C comprises a forward primer comprising a nucleic acid sequence of SEQ ID NO:2, and a reverse primer comprising a nucleic acid sequence of SEQ ID NO:5; and
set D comprises a forward primer comprising a nucleic acid sequence of SEQ ID NO:3, and a reverse primer comprising a nucleic acid sequence of SEQ ID NO:4.
